(19) United States
(12) Patent Application Publication
(10) Pub. No.: US 2011/0003773 A1
(43) Pub. Date: Jan. 6, 2011

(54) USING FULLERENES TO ENHANCE AND STIMULATE HAIR GROWTH

(75) Inventors: Christopher L. Kepley, Ringgold, VA (US); Robert P. Lenk, Danville, VA (US); Stephen R. Wilson, Danville, VA (US); Zhiguo Zhou, Winston-Salem, NC (US)

Correspondence Address: BUCHANAN, INGERSOLL & ROONEY PC, POST OFFICE BOX 1404, ALEXANDRIA, VA, 22313-1404, US

(73) Assignee: Luna Innovations Incorporated, Roanoke, VA (US)

(21) Appl. No.: 12/921,106

(22) PCT Filed: Mar. 3, 2009
(86) PCT No.: PCT/US2009/001334

§ 371 Date: Sep. 3, 2010

Related U.S. Application Data
(60) Provisional application No. 61/033,303, filed on Mar. 3, 2008.

(51) Int. Cl. A61K 31/685; C07C 229/02; A61K 31/215; C07C 69/74; A61P 17/14

(52) U.S. Cl. 514/77; 560/171; 514/529; 560/126

(57) ABSTRACT

Described herein are methods for treating lack of hair growth or a reduction or loss of existing hair by stimulating and/or restoring hair growth, or preventing hair loss. The methods comprise administering to a subject in need thereof a therapeutically effective amount of a fullerene.

### BACKGROUND

Various embodiments described herein relate to the use of fullerenes to treat hair loss and promote hair growth.

Alopecia is the general term referring to any disease, disorder or condition involving hair loss. There are several different types of hair loss, the most common being androgenetic alopecia (AGA; see Sawaya, M. E. Seminars in Cutaneous Medicine and Surgery 17(4):276-283, 1998), alopecia areata (AA; see Fiedler & Alaiti, Dermatologic Clinics 14(4): 733-738, 1996, as well as chemotherapy and drug-induced alopecia. Several hundred diseases have hair loss as a primary symptom.

Alopecia tends to be involuntary and unwelcome. However, it may also be caused by a psychological compulsion to pull out one's own hair or the unforeseen consequences of voluntary hairstyling routines. In some cases, alopecia is an indication of an underlying medical concern, such as iron deficiency or diabetes. Other medical conditions related to hair loss include effluviums, alopecia areata, scarring alopecia, congenital hypotrichosis, infection, and hair shaft defects. Hair loss can also be a side effect of certain therapeutic treatments such as chemotherapy or radiation therapy, or treatment with anticoagulants, medicines used for gout, vitamin A, or antidepressants. Further, hair loss can be related to hormonal changes caused by conditions such as thyroid disease, childbirth, and the use of birth control pills. Significant hair loss has also been observed about three to four months after certain illnesses or major surgery.

Androgenetic alopecia (AGA) is by far the most common type of alopecia. AGA is a patterned, progressive loss of an excessive amount of hair from the scalp. Significant AGA occurs in 50% of men by the age of fifty and 50% of women by the age of sixty. AGA is believed to be a result of both genetic predisposition and the presence of a sufficient level of circulating androgens. It is thought that the enzyme 5 alpha reductase present in dermal papilla cells converts testosterone to dihydrotestosterone (DHT). DHT binds to androgen receptors, also localized in the dermal papilla cells, triggering changes in the hair follicle that result in (1) shortening of the anagen or growth phase of the hair cycle, (2) development of a latent phase in the hair cycle following shedding of the telogen hair, and (3) follicular miniaturization process that reduces the caliber of the anagen hairs produced. It is thought that differential expression of 5-alpha reductase and/or androgen receptors in various types of hair follicles accounts for patterned hair growth and loss.

Hair loss in a defined part of the body is known as alopecia areata. Alopecia areata is a condition primarily affecting humans, in which hair is lost from areas of the body, usually from the scalp. In some cases, the condition can spread to the entire scalp (Alopecia totalis) or to the entire epidermis (Alopecia universalis). Conditions resembling alopecia areata, and having a similar cause, occur also in other species.

Some hair loss conditions go by the name “effluvium.” Effluviums characteristically affect different phases of the hair growth cycle. Telogen effluvium occurs when there is a change in the number of hair follicles growing hair due to an increase in dormant, telogen stage hair follicles. Anagen effluvium is a diffuse hair loss like telogen effluvium, but it develops much more quickly and can cause individuals to lose all their hair. Anagen effluvium is most frequently seen in people taking cytostatic drugs for cancer or those who have ingested toxic products like rat poison.

Alopecia areata is an autoimmune disease where the individual's own immune system attacks hair follicles. Hair loss can also be related to other autoimmune conditions such as lupus.

Hair loss that spreads to cover the entire scalp is called alopecia totalis. If it spreads over the entire body, affecting scalp, eyebrows, lashes, beard, pubic hair, and everything else, then the condition is called alopecia universalis. If the alopecia is just limited to the beard area in men, it is called alopecia barbae.

Scarring alopecia, also known as cicatricial alopecia, refers to a collection of hair loss disorders related to destruction of hair follicles and their replacement with scar tissue. Each specific diagnosis within this category is fairly rare, but some examples include dissecting cellulitis, eosinophilic pustular folliculitis, follicular degeneration syndrome (previously called “hot comb” alopecia), folliculitis decalvans, lichen planopilaris, and pseudopelade of Brocq, to name a few. Scarring alopecia may also be part of a much larger condition such as chronic lupus erythematosus.

Hypotrichosis refers to a condition of no hair growth. Hypotrichoses are conditions that affect individuals from birth and usually stay with them throughout their lives. The majority of hypotrichoses are due to genetic aberrations or defects of embryonic development. There are hundreds of types of genetic hypotrichoses. Often, affected individuals have other physical or mental problems beyond a lack of hair. Conditions such as Graham-Little syndrome, Ofuji syndrome, cartilage-hair hypoplasia, Jeanselme and Rime hypotrichosis, Marie Unna hypotrichosis, and metaphyseal chondrodysplasia, among many others, can involve the symptom of hypotrichosis.

Congenital aplasia, triangular alopecia, congenital atrichia, and monilethrix are other developmental defects that effect hair growth.

Tinea capitis or “ringworm” is a fungal infection that can cause patches of hair loss if it develops on the scalp.

Folliculitis is a term for focal inflammation of hair follicles. In the early stages of a folliculitis the hair fiber may still be present, but as the folliculitis progresses the hair often falls out. When folliculitis is severe, the inflammation can permanently destroy the hair follicles, leaving bald patches. There are forms of folliculitis which are non-infectious such as those caused by oils and greases applied to the skin that clog up the hair follicles, but folliculitis is usually due to a bacterial infection, such as Staphylococcus aureus or Pseudomonas aeruginosa infection. It is also possible to have viral, fungal, or yeast induced folliculitis involving Herpes simplex, Herpes zoster, Pityrosporum ovale, Trichophyton rubrum and other causative agents.

Piedra (Trichomycosis Nodularis) is a condition where the hair fibers are infected by a fungus. There are two basic typres of piedra, black piedra and white piedra, referring to the color of the nodules formed on the hair fiber. Black Piedra is due to the fungus Piedra iahortae and is mostly found in tropical countries while white piedra is due to Trichosporon beigelii and is found mostly in Europe and Southern parts of the USA.

Seborrheic dermatitis is a skin condition that can also involve temporary hair loss if the dermatitis is located on the scalp or other terminal-haired skin areas. Conditions including Parkinson's disease, head injury, and stroke can also be associated with seborrheic dermatitis, and conditions like stress and chronic fatigue can worsen the condition.

In traction alopecia and trichotillomania, the hair is plucked out of the skin leaving clear bald patches or diffuse, thin hair.

Trichorrhexis nodosa is a focal defect in the hair fiber. When observed under the microscope most of a hair shaft looks entirely normal. However, in isolated spots along the length of a fiber swelling and/or fraying can be seen. These focal defects develop where there is an absence of cuticle.

Hair loss can also be related to treatment with cosmetic products, such as excessive shampooing or blow-drying, leading to problems such as overprocessing and cuticle stripping.

Fullerene molecules are a family of carbon allotropes that comprise closed cages of generally 20 to 200 carbon atoms and may also include chemical moieties attached to the exterior or incorporated within the cage. Fullerenes can be in the form of a hollow sphere, ellipsoid, or tube. The most common fullerene to date is the C₆₀ Buckminsterfullerene (IUPAC name (C60-Ih)[5,6]fullerene). Another fairly common buckminsterfullerene is C₇₀, but fullerenes with 72, 76, 84 and even up to 100 carbon atoms are commonly obtained. Fullerene molecules can contain 500 or more carbon atoms.

Structural variations include nonclosed-cage structures, heterofullerenes, derivatives formed by substitution of hydrofullerenes, the fusion of organic rings or ring systems to the fullerene cage, chiral fullerenes, buckyball clusters, nanotubes, megatubes, polymers, nano “onions,” linked “ball-and-chain” dimers, and fullerene rings (see, e.g., Miessler and Tarr (2004) Inorg. Chem. 3, Pearson Education International. ISBN 0-13-120198-0; Mitchel et. al. (2001) Inorg. Chem. 40: 2751; Sano (2001) Nature (London) 414: 506; Shvartsburg (1999) Phys. Chem. 103: 5275; and Li et al. (2001) Chem. Phys. Lett. 335: 524).

In general, fullerenes are hydrophobic and sparingly soluble in many solvents (see, e.g., Braun, et al., Fullerenes, Nanotubes and Carbon Nanostructures 15;311-314, 2007). However, a variety of procedures for functionalizing fullerenes are known in the art, and some of the derivative fullerenes are water soluble (see, e.g., U.S. Pat. No. 5,648,243 to Chiang; U.S. Patent Application Publication Nos. 2008/0004345 and 2004/0044062; Jensen et al., Bioorganic & Medicinal Chemistry, 4:767-79, 1996; Da Ros et al., Croatica Chemica Acta CCACAA 74:743-55 (2001); Wilson, in “Perspectives in Fullerene Nanotechnology,” Osawa, ed., (Kluwer Academic Publishers, Dorcrecht, Netherlands, 2000); Syrensky, et al., Kopf Carrier #63, (David Kopf Instruments Tujunga, California, September 2006); Y. L. Lai and L. Y. Chiang, J. Autonomic Pharmacol., 17:229, 1997; Schinazi et al., Proc. Electrochem. Soc., 97:10, 1997; Lai et al., World J. Surg., 24:450, 2000; Jin et al., J. Neuroscience Res., 62:600, 2000; Huang et al., Free Radical Biol. Med., 30:643, 2001; Chi et al., in “Perspectives of Fullerene Nanotechnology,” pp 165-183, E. Osawa ed., (Kluwer Academic Publisher, Great Britain, 2002); Dugan et al., P.N.A.S. 94:9434-39, 1997; Dugan et al., Parkinsonism & Related Disorders 7:243-46, 2001; Quick et al., Neurobiol of Aging (electronic publication 2006); Kato et al., Chem & Biodiv., 2:1232-1241, 2005; and Georgakilas et al, Proc. Nat. Acad. Sci. 99;5075-5080, 2002).

Incorporation of fullerenes into lipid vesicles has also been studied (see, e.g., Bensasson et al. (Journal of Physical Chemistry, 98:3492-3500, 1994); Hirsch et al. (Angewandte Chemie International Edition 39:1845-1848, 1999); U.S. Pat. No. 7,070,810; and Felder, et al., Hely. Chim. Acta, 85: 288-319, 2002)

Fullerenes can also be modified at their surface to present specific biologically active groups, such as lectins or antibodies (see, e.g., U.S. Patent Application Publication No. 2005/0043787 and U.S. Pat. No. 5,310,669). Certain chemically modified fullerenes are commercially available (see, e.g., BuckyUSA, Houston, Tex. and American Dye Source, Inc., Quebec, Canada). Fullerenes and fullerene derivatives have been proposed as free radical scavengers (see, e.g., Haddon (J. Am. Chem. Soc. 112:3389, 1990); U.S. Pat. No. 5,648,243 to Chiang, U.S. Patent Application Publication No. 2003/0162837 by Dugan, U.S. Pat. No. 7,163,956 to Wilson; and Kepley (J. Immunol. 179:665, 2007). U. S. Patent Application No. 2005/0058675 describes the use of fullerene derivatives to treat dermatological conditions due to injuries caused by oxidative stress, including sunburn and ionizing radiation. Reference is made to conditions such as sunburn, aging, hair loss, psoriasis, acne or smoker's face.

### SUMMARY

It has been discovered that fullerenes can stimulate hair growth, restore hair growth in areas of hair loss, and induce the formation of new hair follicles. Described herein are methods for treating lack of hair growth or a reduction or loss of existing hair by stimulating and/or restoring hair growth, or preventing hair loss, comprising administering to a subject in need thereof a therapeutically effective amount of a fullerene. The administering may be repeated as necessary or desired to result in a desired level of hair growth and/or prevention of hair loss. The methods may be practiced with any fullerene, which may be delivered systemically or locally.

In one embodiment, a method of preventing hair loss and/or promoting hair growth, comprising administering a therapeutically effective amount of a fullerene to a subject in need thereof, is provided. The fullerene can be administered to an area of skin exhibiting or suspected of hair growth reduction or hair loss. In certain embodiments, the fullerene is injected at the area, or is administered topically. The fullerene can be derivatized.

### BRIEF DESCRIPTION OF THE DRAWINGS

FIG. 1 illustrates non-limiting examples of water insoluble and water soluble fullerenes.

FIG. 2 illustrates hair growth enhancement in shaved C57/B6 mice treated with compound 5.

FIG. 3 illustrates hair growth enhancement in shaved C57/B6 mice treated with Polyhydroxy-C₆₀.

FIG. 4 illustrates the hair follicle generation in SKH-1 bald mice treated with compound 5.

FIG. 5 illustrates the structures of Compound 5 (C₇₀-ALM) and 7 (C₇₀-TGA).

FIG. 6 illustrates the structures of exemplary but non-limiting fullerene derivatives, including Compound 10 (C₇₀-tetrainositol) and 12 (C₇₀-TEG acid).

FIG. 7 illustrates the structures of exemplary but non-limiting fullerene derivatives, including Compound 13 (C₇₀-ALM-GSH).

FIG. 8 illustrates the structure and synthesis of Compound 12 (C₇₀-ALM PC).

FIG. 9 illustrates the structure and synthesis of Compound 23 (C₇₀-ALM inositol).

FIG. 10 (top row) illustrates the difference in hair growth in depilated mice between mice treated with placebo (left) or compound 7 (right). Micrographs of sections of the skin of these mice (bottom row) demonstrate the increase in number of follicles in the mice treated with compound 7 (right) as compared to placebo (left).

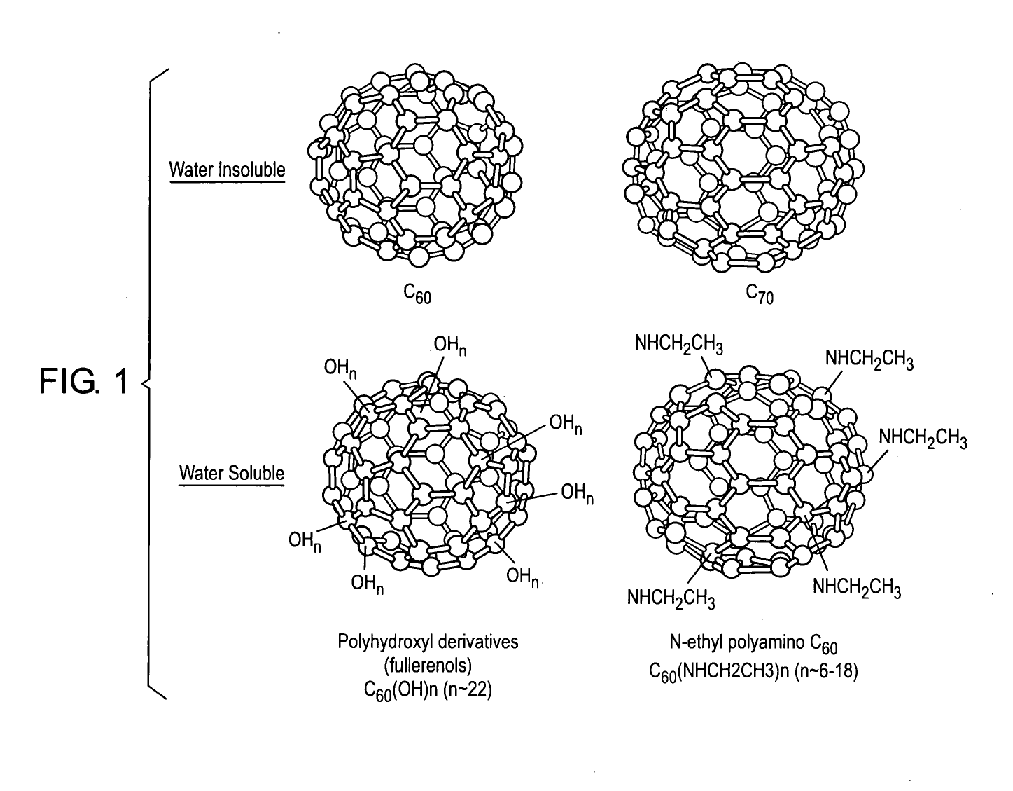

### DETAILED DESCRIPTION

In accordance with this detailed description, the following definitions apply.

As used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The methods are related to enhancing, stimulating, inducing, promoting, restoring, reviving, renewing, replacing or otherwise activating hair growth in animals characterized by a lack of hair growth, inhibition of hair growth, or a reduction in the amount of, or loss of, hair. The methods described herein can be used to stimulate hair growth and to prevent hair loss in any situation in which additional hair growth and/or decreased hair loss is desired.

In particular, the present methods will be useful when the subject has experienced loss of hair associated with a variety of conditions, including, but not limited to the following: androgenetic alopecia, alopecia areata, drug-induced alopecia (for example following chemotherapy treatment for cancer), hair loss due to radiation treatment, anagen effluvium, drug-induced alopecia, radiotherapy, poisoning, diffuse alopecia areata, alopecia areata, loose anagen syndrome, postoperative occipital alopecia, syphilis, traction alopecia, tricholtillomania tinea capitis, telogen effluvium, telogen gravidarum, chronic telogen effluvium, early androgenentic alopecia, iron deficiency, malnutrition/malabsorption, hypothyroidism, hyperthyroidism, systemic lupus erythematosus, chronic renal failure, hepatic failure, advanced malignancy, folliculitis, seborrheic dermatitis, and viral, fungal or bacterial infection.

“Fullerene” or “fullerene molecule” as used herein refers to any member of the fullerene family of carbon cage molecules. Fullerenes are generally carbon structures formed of five and six membered rings arranged so that the rings form a closed geodesic sphere or spheroid held together by a combination of single and double carbon:carbon covalent bonds. The fullerenes in this disclosure can be defined by the formula: C₂ₛ wherein s is greater than or equal to 30, such as from about 30 to about 200 or from about 30 to about 100. For example, the fullerenes include C₆₀, C₇₀, and similar molecules that range in molecular weight from C₆₀ up to C₈₄, C₉₀, and larger such molecules, with shapes ranging from spheroidal to ellipsoidal, elongated and other shapes, and including not only single-walled but also multi-walled cages consisting of stacked or parallel layers. The fullerenes may be unmodified or underivatized. Alternatively, the fullerenes may enclose one or more atoms such as metal atoms, or other small chemical groups, inside the carbon cage; such fullerenes are sometimes called endohedral fullerenes. Fullerenes, as used herein, also includes structures with chemical functional groups attached to the surface of the carbon cage. The functional groups can be covalently bound to the carbon cage via opening carbon:carbon double bonds. Fullerenes also include other structural variants, derivatives, and/or modified or functionalized fullerenes as described herein and/or as known in the art. The fullerenes can be synthetic or naturally-occurring. Synthetic fullerene molecules can be prepared in a laboratory by known methods (see, e.g., U.S. Pat. No. 5,177,248 and Kratschmer et al., Chem. Phys. Lett., 170, 167-170 (1990)) or can be purchased commercially.

In one embodiment, the fullerenes are water soluble, meaning the fullerenes distribute more or less uniformly in an aqueous solution and do not significantly precipitate. Water soluble fullerenes are known in the art as described above, and can be synthesized for example by attaching one or more hydrophilic chemical groups to the surface of the carbon cage. Suitable hydrophilic chemical groups include hydroxyl or polyhydroxyl groups and N-palyaminoethyl groups. Non-limiting examples of water soluble fullerenes include C₆₀(OH)ₙ, C₆₀(NH—CH₂—CH₃)ₙ, and compounds 7 10 and 12. Many other examples of water-soluble fullerenes are known and can involve the addition of one or more polar groups such as phosphates, sulfates, ammonium, carboxylates, or other charged groups; hydroxyl and polyhydroxyl groups; and carbohydrates, peptides, proteins, and DNA.

In an exemplary embodiment, chemical groups such as amphiphilic or lipophilic groups can be attached to the carbon cage instead of or in combination with hydrophilic chemical groups.

“Fullerene” or “fullerene molecule” as used herein refers to certain synthetically modified fullerene molecules as described herein, including amphiphilic or lipophilic synthetically modified fullerenes of the formula Zₘ—F—Yₙ; and hydrophilic or amphiphilic synthetically modified fullerenes of the formula Z′ₘ—F—Y′ₙ. The fullerenes comprise closed cages of 60 to 200 carbon atoms which may also include chemical moieties attached to the exterior and/or incorporated within the cage.

The amphiphilic or lipophilic synthetically modified fullerene molecules are described in copending U.S. patent application Ser. No. 2/073,230, U.S. Patent Application Publication No. 2008-0213324-A1, filed Mar. 3, 2008, entitled “AMPHIPHILIC OR LIPOPHILIC POLAR FUNCTIONALIZED FULLERENES AND THEIR USES,” the entire disclosure of which is incorporated by reference herein.

The amphiphilic or lipophilic and hydrophilic or amphiphilic synthetically modified fullerene molecules as described in the copending application include fullerenes that, have an aspect ratio ≠1, with an equatorial band and two opposing poles, and comprise an adduct at one or both poles.

In one embodiment, the amphiphilic or lipophilic synthetically modified fullerene has the formula

Zₘ—F—Yₙ;

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage.

Z and Y are positioned near respective opposite poles of Cₚ;

m=1-5 and Z is a hydrophilic, lipophilic, or amphiphilic chemical moiety;

n=1-5 and Y is a lipophilic chemical moiety;

p=60-200 and p is an even number; and

X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The synthetically modified fullerene can be arranged wherein each chemical moiety Z is composed of formula AᵣB in which A is a hydrophilic, lipophilic or amphiphilic chemical moiety, r=1-4, and B is a chemical linker connecting said A to the fullerene, and each chemical moiety Y is composed of formula DEᵥ in which E is a lipophilic chemical moiety, v=1-4, and D is a chemical linker connecting the lipophilic chemical moiety to the fullerene.

The amphiphilic or lipophilic synthetically modified fullerene can be a prolate ellipsoid shaped fullerene having a major axis such that said poles are located at opposing ends of the major axis of the prolate ellipsoid fullerene. Alternatively, the fullerene can be spheroid with opposing poles defined by an axis through opposing carbon rings. Z and Y can configured such that when the molecule is contacted with a lipid bilayer in an aqueous medium, the equatorial region of F is selectively located within or in close proximity to the phospholipid bilayer. The molecule can be configured so that in an extended configuration has an aspect ratio of about 2.1 to 15, and a diameter less than about 2 nm. Such configurations are preferred configurations for incorporation of the molecules into lipid bilayers.

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y; and, wherein said lipophilic moiety Y is capable of anchoring the synthetic fullerene molecule to a lipid membrane;

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y; wherein Y is a lipophilic moiety covalently connected to C₇₀, optionally through a linking group, at or near a pole thereof, and wherein Z is a lipophilic moiety, amphiphilic moiety, or a hydrophilic moiety covalently connected to C₇₀, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y wherein: Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane,

In another embodiment the amphiphilic or lipophilic synthetically modified fullerene molecule can have the formula Zₘ—F—Yₙ wherein:

F is a fullerene of formula Cₚ having p=60-200 carbons, preferably p=60 or 70;

m=1-5 such that each Z is a group AᵣBₛ in which r=1-4, s=1-4, and A is one or more hydrophilic or polar group bonded to the fullerene through one or more linker B;

n=1-5 and each Y is a group DₜEᵥ in which t=1-4, v=1-4 and E is one or more lipophilic group bonded to the fullerene through one or more linker D; and,

X and Y are positioned at or near opposite poles of F.

Examples of amphiphilic synthetically modified fullerene molecules with a lipid group on one pole of the fullerene and a hydrophilic group on the other pole are compounds 5, 13, 21, and 23.

In certain embodiments the amphiphilic or lipophilic synthetically modified fullerene has a geometrical configuration capable of causing the fullerene molecule to locate within phospholipid bilayers of a cell such that a radical scavenging zone near the equatorial band of the fullerene is situated within or in close proximity to the phospholipid bilayer.

A plurality of such synthetically modified fullerene molecules can be uniformly dispersed in phospholipids, such as in liposomes. The amphipathic fullerene molecules described herein do not generally form vesicles by themselves, but require membrane-forming phospholipids in mole ratios greater than 1:1 (lipid:fullerene adduct) to form vesicles.

The methods described herein also encompass hydrophilic or amphiphilic synthetically modified fullerenes of the formula

Z′ₘ—F—Y′ₙ;

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage;

Z′ and Y′ are positioned near respective opposite poles of Cₚ;

m=1-5 and Z′ is a hydrophilic, lipophilic, or amphiphilic chemical moiety;

n=1-5 and Y′ is a hydrophilic or amphiphilic chemical moiety;

p=60-200 and p is an even number; and

X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The fullerene can be arranged wherein each chemical moiety Z′ is composed of formula A′ᵣB in which A′ is a hydrophilic, lipophilic or amphiphilic chemical moiety, r=1-4, and B is a chemical linker connecting said A′ to the fullerene, and each chemical moiety Y′ is composed of formula DE′ᵥ in which E′ is a hydrophilic or amphiphilic chemical moiety and, v=1-4, and D is a chemical linker connecting the chemical moiety Y′ to the fullerene.

In another embodiment, the hydrophilic or amphiphilic synthetically modified fullerene molecule has the formula Z′(Cₚ)Y′ wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

In exemplary embodiments, Z′ and Y′ are both amphiphilic; Z′ and Y′ are both hydrophilic; or one of Z′ and Y′ is amphiphilic while the other is hydrophilic. In other embodiments, Z′ is lipophilic and Y′ is hydrophilic or amphiphilic.

In another embodiment, the hydrophilic or amphiphilic synthetically modified fullerene molecule has the formula Z′(C₇₀)Y′; wherein Y′ is a hydrophilic or amphiphilic moiety covalently connected to C₇₀, optionally through a linking group, at or near a pole thereof, and wherein Z′ is a hydrophilic or amphiphilic moiety covalently connected to C₇₀, optionally through a linking group, at or near a pole opposite to said Y′. Examples of synthetically modified fullerene molecules with hydrophilic groups at each pole include compounds 7, 10, and 12.

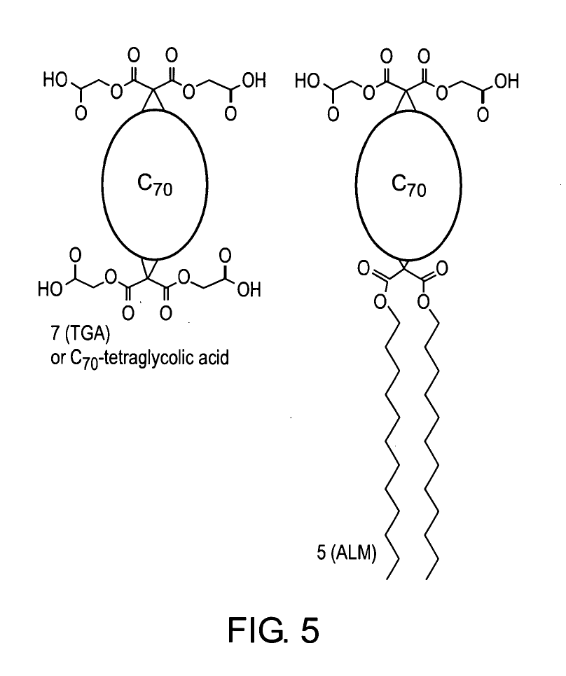

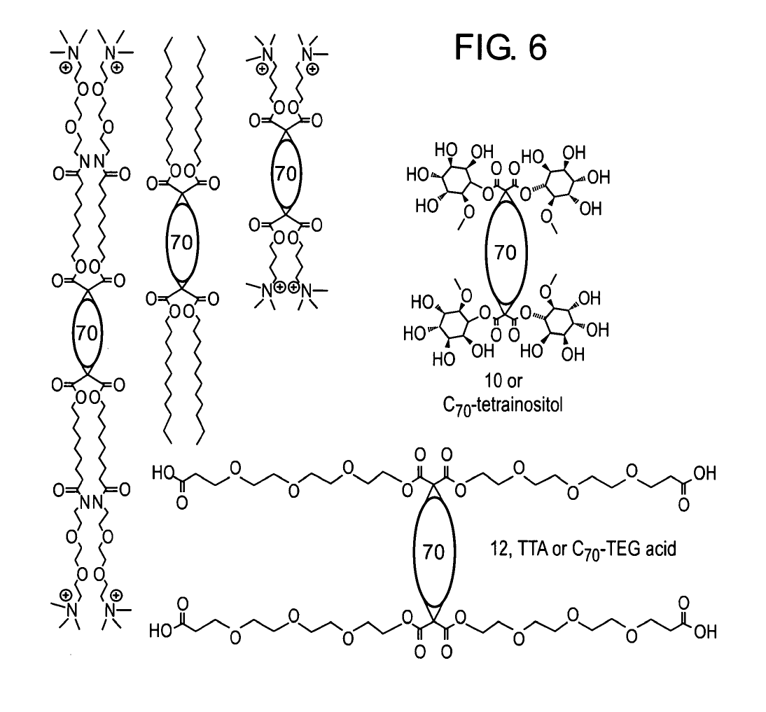

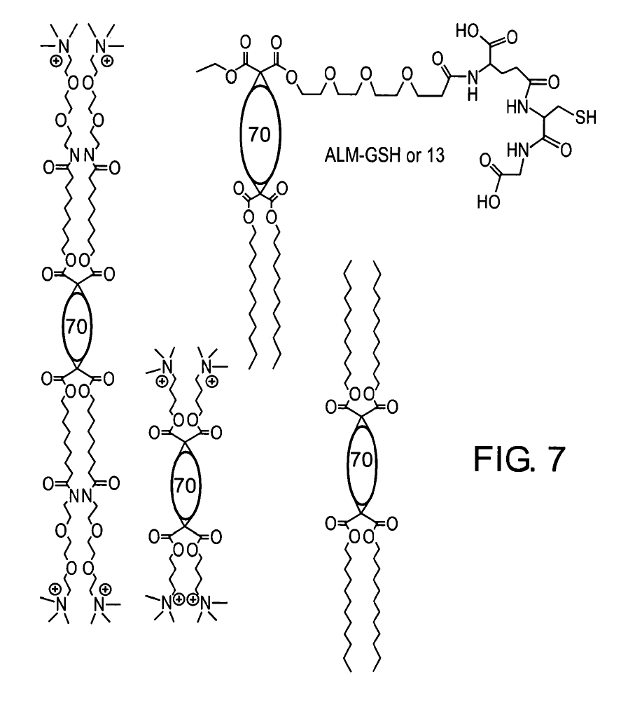

In certain embodiments, the fullerene comprises a fullerene of compound 5 (see FIG. 5). In the present examples, compound 5 comprises C₇₀. In other embodiments the fullerene is one or more of the additional compounds shown in the present FIGS. 5, 6, 7, 8 and 9, such as compounds 7, 10, 12, 13, 21, and/or 23. In yet another embodiment, the fullerene is Polyhydroxy-C₆₀.

Suitable fullerenes are also described in the following co-pending PCT applications filed concurrently herewith: Attorney Docket No. 1034136-000063, entitled “METHOD FOR TREATING PRURITUS BY ADMINISTERING FULLERENES;” Attorney Docket No. 1034136-000064, entitled “FULLERENE THERAPIES FOR INFLAMMATION;” Attorney Docket No. 1034136-000065, entitled “METHOD FOR INHIBITING THE BUILD-UP OF ARTERIAL PLAQUE;” and Attorney Docket No. 1034136-000066, entitled “METHOD FOR TREATING WOUNDS BY ADMINISTERING FULLERENES;” the entire disclosures of which are incorporated by reference herein, and in U.S. Patent Application Nos. 61/071,756, filed May 15, 2008, entitled “NEW REACTIONS OF FULLERENES” and Ser. No. 12/073,231, filed Mar. 3, 2008, entitled “STEROID DERIVATIVES OF FULLERENES,” the entire disclosures of which are incorporated by reference herein.

“Pharmaceutically acceptable salt” refers to pharmaceutically acceptable salts of fullerenes which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The terms “treating,” “treatment,” and the like are used herein to generally mean obtaining a desired pharmacological and physiological effect, and refer to complete elimination as well as to any clinically or quantitatively measurable reduction in the hair loss for which the subject is being treated, as well as to any clinically or quantitatively measurable enhancement of hair growth.

A “subject in need thereof” refers to any subject or individual who could benefit from the method of treatment described herein. In certain embodiments, a subject in need thereof is a subject predisposed for the development of hair loss; a subject having one or more disorders related to hair loss but not exhibiting any clinical symptoms; and/or a subject exhibiting hair loss. The hair loss can be partial or total, and can be over the entire body or only in one or more discrete areas of the body.

The “subject in need thereof” refers to a vertebrate, such as a mammal. Mammals include, but are not limited to, humans, other primates, rodents (i.e., mice, rats, and hamsters), farm animals, sport animals and pets. In one embodiment, the subject is a mammal such as a human. In certain embodiments, the methods find use in experimental animals, in veterinary application, and/or in the development of animal models for disease.

A “therapeutically effective amount” or “pharmaceutically effective amount” means the amount of a fullerene that, when administered to a subject for enhancing hair growth and/or inhibiting hair loss, is sufficient to effect such treatment. Thus a “therapeutically effective amount” is an amount indicated for treatment while not exceeding an amount which may cause significant adverse effects. The “therapeutically effective amount” will vary depending on the fullerene, and will also be determined by physical and physiological factors such the age, body weight, and/or clinical history of the subject to be treated. Methods for evaluating the effectiveness of therapeutic treatments are known to those of skill in the art.

In one embodiment, the methods described herein provide for the use of fullerenes to stimulate an increase in hair count numbers and restore hair growth in areas of hair loss. The hair loss can be total or partial. In one embodiment, the increase in hair count corresponds to an increase in terminal hairs, which are long, pigmented hairs that are produced by follicles with sebaceous (oil) glands. They are found on the scalp, beard, armpits and pubic areas and are in contrast to vellus hairs, which are short hairs, often only a centimeter or two long, that contain little or no pigment. The follicles that produce vellus hairs do not have sebaceous and never produce any other kind of hairs. Terminal hairs also differ from Lanugo hair, which develops on an unborn baby. In people who have inherited a tendency to baldness terminal hairs may gradually become thinner and shorter until they look like vellus hairs. This may be due to the growth of terminal hairs being influenced by hormones (e.g. androgens) while vellus hairs are not so influenced.

The progression of conditions such as AGA generally comprise a gradual decrease in the number of terminal hairs over time. The terminal hairs may also gradually become thinner and shorter until they look like vellus hairs. In addition to treatment of hair loss, the methods described herein may be used for the stimulation of hair growth in areas not recognized as experiencing hair loss. As used herein, the term “hair growth” comprises an increase in number of terminal hairs present. Terminal hair counts can be conducted in a number of ways as known in the art. A non-limiting example is where the terminal hair is counted by trained and validated technicians who perform a computer-assisted count on macrophotographs (see, e.g., Canfield, Dermatologic Clinics, 14:713-721 (1996)). In brief, a target area on the scalp is chosen, the hair clipped and the scalp permanently marked with a single dot tattoo in the center in order to facilitate the exact positioning at each subsequent photo session. The macrophotography is performed using a preset camera with a macro lens and a stand that provides a constant reproduction ratio and electronic flashes that reproducibly illuminate the area to photograph. The images are taken in triplicate, centering the camera using the tattoo and the color slide films are processed at a central facility. The quality of the images is assessed and large transparencies are made of the best images. The terminal hairs on the target circle of the transparencies are then counted by the trained technicians.

In certain embodiments, the methods described herein provide between 1% to 100% increase in the numbers of new hair follicles and terminal hairs within 12 to 90 days of the fullerene treatment. As shown in FIG. 5, the methods may provide at least a 75% increase in the number of hair follicles within 14 days of the fullerene treatment.

The fullerenes may be formulated into a variety of compositions (i.e. formulations or preparations). These compositions may comprise any component that is suitable for the intended purpose, such as conventional physiologically acceptable delivery vehicles, diluents and excipients including isotonising agents, pH regulators, solvents, solubilizers, dyes, gelling agents and thickeners and buffers and combinations thereof. Pharmaceutical formulations suitable for use with the instant fullerenes can be found, for instance, in Remington's Pharmaceutical Sciences. Physiologically acceptable carriers are carriers that are nontoxic at the dosages and concentrations employed. Pharmaceutical formulations herein comprise pharmaceutical excipients or carriers capable of directing the fullerenes to the area of hair growth reduction or hair loss. Suitable excipients for use with fullerenes include water, saline, dextrose, glycerol and the like.

In certain embodiments, the formulations comprise a skin-penetration enhancer. Any skin-penetration enhancer suitable for aiding the delivery of the fullerenes can be used. A list of skin-penetration enhancers can be found in, e.g., “Pharmaceutical Skin Penetration Enhancement” (1993) Walters, K. A., ed.; Hadgraft, J., ed—New York, N.Y. Marcel Dekker and in “Skin Penetration Enhancers cited in the Technical Literature” Osbourne, D. W. Pharmaceutical Technology, November 1997, pp 59-65.

The formulations can comprise from about 0.1% to about 99%, such as from about 0.1% to about 90%, about 5% to about 90%, or about 15% to about 75%, by weight of skin penetration enhancer. In certain embodiments the ratio of fullerenes to skin-penetration enhancer is from about 1:20 to about 1:10000, such as from about 1:60 to 1:300, on the basis of percentages by weight of total composition.

The fullerene may be solubilized, especially when the fullerene is hydrophobic. One method of solubilizing certain fullerenes is by formulation in liposomes. Other methods suitable for solubilizing certain fullerenes include the use of a solvent acceptable for use in the treatment of skin tissues and cells such as, but not limited to, dimethylsulfoxid (DMSO), polyethylene glycol (PEG), such as PEG 1000 or less, or any other solvent. Other solubilizers include glycol ethers, polyethylene glycol derivatives, propylene glycol, propylene glycol derivatives, polysorbates (e.g. TWEEN™), fatty alcohols, aromatic alcohols, propylene glycol, glycerols, oils, surfactants, glucosides, and mixtures thereof. In certain embodiments the solubilizer is selected from diethylene glycol monoethyl ether (TRANSCUTOL®), polyethylene glycol of average molecular weight from 100 to 1000, triethylene glycol, tetraethylene glycol, pentaethylene glycol, hexaethylene glycol, septaethylene glycol, octaethylene glycol, propylene glycol, propylene glycol mono- and diesters of fats and fatty acids (e.g. propylene glycol monocaprylate, propylene glycol monolaurate), benzyl alcohol, glycerol, oleyl alcohol, mineral oil, lanolin/lanolin derivatives, petrolatum or other petroleum products suitable for application to the skin, propylene glycol mono- and diesters of fats and fatty acids, macrogols, macrogolglycerides or polyethylene glycol glycerides and fatty esters (e.g. stearoyl macrogolglycerides, oleoyl macrogolglycerides, lauroyl macrogolglycerides, linoleoyl macrogolglycerides), ethoxylated castor oil (e.g. Cremophor—a polyoxyl hydrogenated castor oil), C6-C30 triglycerides, natural oils, glucosides (e.g. cetearyl glucoside), surfactants, and mixtures thereof. In certain embodiments the formulations herein comprise from about 0.1% to about 99% by weight of solubilizer, such as from about 1% to about 75% by weight of solubilizer.

In certain embodiments the formulations have a viscosity at 20° C. of from about 50 cps to about 50000 cps, such as from about 500 cps to about 40000 cps, or about 5000 cps to about 30000 cps. Should the viscosity need to be adjusted it can be done by means of a viscosity modifying agent. Examples of viscosity modifiers include polyethylene glycols, acrylic acid-based polymers (carbopol polymers or carbomers), polymers of acrylic acid crosslinked with allyl sucrose or allylpentaerythritol (carbopol homopolymers), polymers of acrylic acid modified by long chain (C₁₀-C₃₀) alkyl acrylates and crosslinked with allylpentaerythritol (carbopol copolymers), poloxamers also known as pluronics (block polymers; e.g. Poloxamer 124, 188, 237, 338, 407), waxes (paraffin, glyceryl monostearate, diethylene glycol monostearate, propylene glycol monostearate, ethylene glycol monosterate, glycol stearate), hard fats (e.g. Saturated C₈-C₁₈ fatty acid glycerides), xantham gum, polyvinyl alcohol, solid alcohols, and mixtures thereof.

In certain embodiments the formulations contain one or more PEGs. Examples include at least one PEG of average molecular weight about 2000 or less, about 1500 or less, about 1000 or less, about 800 or less, about 600 or less, about 500 or less, or about 400 or less. Examples also include at least one PEG of average molecular weight about 3000 or more, about 3350 or more, or about 3500 or more. In one embodiment the formulation comprises a mixture of PEG's, such as at least one PEG having an average molecular weight of about 800 or less and at least one PEG having an average molecular weight of about 3000 or more.

The formulation may comprise a variety of other components. Any suitable ingredient may be used herein but typically these optional component will render the formulations more cosmetically acceptable or provide additional usage benefits. Some examples of optional ingredients include, but are not limited to, emulsifiers, humectants, emollients, surfactants, oils, waxes, fatty alcohols, dispersants, skin-benefit agents, pH adjusters, dyes/colourants, analgesics, perfumes, preservatives, and mixtures thereof. In addition, the methods described herein include use of combination compositions comprising the fullerenes as described herein in combination with other agents suitable for the promotion of hair growth and/or treatment of hair loss.

Examples of suitable preservatives include but are not limited to parabens, benzyl alcohol, quaternium 15, imidazolidyl urea, disodium EDTA, methylisothiazoline, alcohols, and mixtures thereof. Examples of suitable emulsifiers include but are not limited to waxes, sorbitan esters, polysorbates, ethoxylated castor oil, ethoxylated fatty alcohols, macrogolglycerides or polyethylene glycol glycerides and fatty esters (e.g. stearoyl macrogolglycerides, oleoyl macrogolglycerides, lauroyl macrogolglycerides), esters of saturated fatty acids (e.g. diethylene glycol parmitostearate), macrogols of cetostearyl ether (e.g. macrogol-6-cetostearyl ether), polymers of high molecular weight, crosslinked acrylic acid-based polymers (carbopols or carbomers), and mixtures thereof. Examples of suitable emollients include but are not limited to propylene glycol dipelargonate, 2-octyldodecyl myristate, non-polar esters, triglycerides and esters (animal and vegetable oils), lanolin, lanolin derivatives, cholesterol, glucosides (e.g. cetearyl glucoside), pegylated lanolin, ethoxylated glycerides, and mixtures thereof. Examples of suitable surfactants include but are not limited to sorbitan esters, polysorbates, sarcosinates, taurate, ethoxylated castor oil, ethoxylated fatty alcohols, ethoxylated glycerides, caprylocaproyl macrogol-8 glycerides, polyglyceryl-6 dioleate, and mixtures thereof. Examples of suitable oils include but are not limited to propylene glycol monocaprylate, medium chain triglycerides (MCT), 2-octyl-dodecyl myristate, cetearyl ethylhexanoate, and mixtures thereof. Examples of suitable fatty alcohols include but are not limited to cetostearyl alcohol, cetyl alcohol, stearyl alcohol, and mixtures thereof. Also useful in the formulations herein are lipids and triglycerides (e.g. concentrates of Seed Oil Lipids, Concentrates of Marine Oil Lipids, high purity triglycerides and esters), alkyl ether sulfates, alkyl polyglycosides, alkylsulfates, amphoterics cream bases, and mixtures thereof.

Preparation of dry formulations that are reconstituted immediately before use also is contemplated. The preparation of dry or lyophilized formulations can be effected in a known manner, conveniently from fullerene solutions as described herein. The dry formulations are also storable. By conventional techniques, a solution can be evaporated to dryness under mild conditions, especially after the addition of solvents, such as a mixture of toluene and ethanol, for azeotropic removal of water. The residue is thereafter conveniently dried, e.g. for some hours in a drying oven.

The methods described herein are targeted to hair follicles and/or surrounding tissues and cells as a treatment for alopecia. The fullerene-containing preparations described above may be administered systemically or locally and may be used alone or as components of mixtures. In one embodiment the administration is local. The route of administration for the fullerenes may be topical, intradermal, intravenous, oral, or by use of an implant. In one embodiment the route of administration is topical. For example, fullerenes may be administered by means including, but not limited to, topical lotions, topical creams, topical pastes, topical suspensions, intravenous injections or infusions, oral intake, or local administration in the form of intradermal injection or an implant. Additional routes of administration are subcutaneous, intramuscular, or intraperitoneal injections of the fullerenes in conventional or convenient forms.

Suitable isotonising agents are for example nonionic isotonising agents such as urea, glycerol, sorbitol, mannitol, aminoethanol or propylene glycol as well as ionic isotonising agents such as sodium chloride. Solutions containing fullerenes will contain the isotonising agent, if present, in an amount sufficient to bring about the formation of an approximately isotonic solution. The expression “an approximately isotonic solution” will be taken to mean in this context a solution that has an osmolarity of about 300 milliosmol (mOsm), conveniently 300+10% mOsm. It should be borne in mind that all components of the solution contribute to the osmolarity. The nonionic isotonising agent, if present, is added in customary amounts, for example in amounts of about 1 to about 3.5 percent by weight, such as in amounts of about 1.5 to 3 percent by weight.

In one embodiment, the fullerenes are delivered topically. For topical administration, the fullerenes may be in standard topical formulations and compositions including lotions, creams, suspensions, serums, or pastes. Solubilized fullerenes can also be added to other dermatological products, such as hair gels, shampoos, conditioners, styling products, soaps, or the like. Injection may also be used when desired. Oral administration of suitable formulations may also be appropriate in those instances where the fullerenes may be readily administered to the hair follicle and/or surrounding tissues or cells via this route.

Modes of administration can include delivery via a sustained release and/or controlled release drug delivery formulation and/or device. “Sustained release” refers to release of a drug or an active metabolite thereof into the systemic circulation over a prolonged period of time relative to that achieved by oral administration of a conventional formulation of the drug. “Controlled release” is a zero order release; that is, the drug releases over time irrespective of concentration. Single, multiple, continuous or intermittent administration can be effected.

The dose can be determined by one of skill in the art without an undue amount of experimentation. The dose of fullerene may be optimized by the skilled artisan depending on factors such as, but not limited to, the fullerene chosen, the physical delivery system in which it is carried, the individual subject, the underlying disorder and the severity of the disorder, the treatment period, frequency of administration, and the judgment of the skilled practitioner. For topical formulations (such as ointments) to be applied to the surface of the skin, exemplary concentrations of the fullerenes in the excipient range from about 0.001 to about 10% w/w, such as from about 0.005 to about 5% w/w, or from about 0.01 to about 1% w/w. The foregoing ranges are merely suggestive in that the number of variables with regard to an individual treatment regime is large and considerable deviation from these values may be expected. When administered topically, the area to be treated may be massaged after application of the fullerenes.

In one embodiment, the fullerenes are delivered intradermally. For intradermal administration, the fullerenes may be in standard injectable formulations and compositions including suspensions or serums.

The fullerenes may be applied to a small or large area of the body or scalp depending on the area to be treated. The fullerenes may be applied to the skin and/or to the hair of the subject. The fullerene treatment may comprise a single administration. Alternatively, the treatment may be repeated. The frequency of treatment may vary. For example, the treatments could be daily, every two days, twice weekly, weekly, ever two weeks, twice monthly, every four weeks, monthly, every six weeks, every eight weeks, every two months, quarterly, twice annually, or annually, or other suitable time interval to stimulate hair growth, prevent hair loss, and/or maintain the prevailing condition. In one embodiment, the treatment is repeated at least once every six months, such as at least once every three months or at least once every two months.

In certain embodiments, such as cases where hair loss is observed, maintenance treatment on a regular basis may be initiated and sustained. The total number of treatments in any 3 month period can be from 1 to 90. The mode of administration and dosage may be readily adjusted using routine experimentation to produce a desired level of alopecia treatment without causing significant damage to the surrounding tissue. While not wishing to be bound by theory, it is believed that different fullerenes, different formulations, and different modes of administration will require different parameters in order to cause hair growth. Such parameters can be determined by simple dose-ranging studies. For example, a suitable method could involve: (a) taking a terminal hair count, (b) applying the fullerene compositions at various strengths, (c) waiting for varying lengths of time, and (e) reassessing hair counts. Alternatively, the study might involve some other methods of assessing hair growth such as (visual) assessment of the hair density, hair weight, and/or hair shaft diameter.

The present disclosure relates to use of any one or more of the fullerenes described herein for preventing hair loss and/or promoting hair growth. The present disclosure also relates to use of any one or more of the fullerenes described herein for the manufacture of a medicament, particularly for the manufacture of a medicament for preventing hair loss and/or promoting hair growth.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications, patents, patent applications and other references cited herein are hereby incorporated by reference.

While the disclosure has been described in detail with reference to certain embodiments thereof, it will be apparent to one skilled in the art that various changes can be made, and equivalents employed, without departing from the scope of the disclosure. In addition, the following examples are illustrative of the methods described herein and should not be considered as limiting the foregoing disclosure in any way.

### Examples

### Example 1

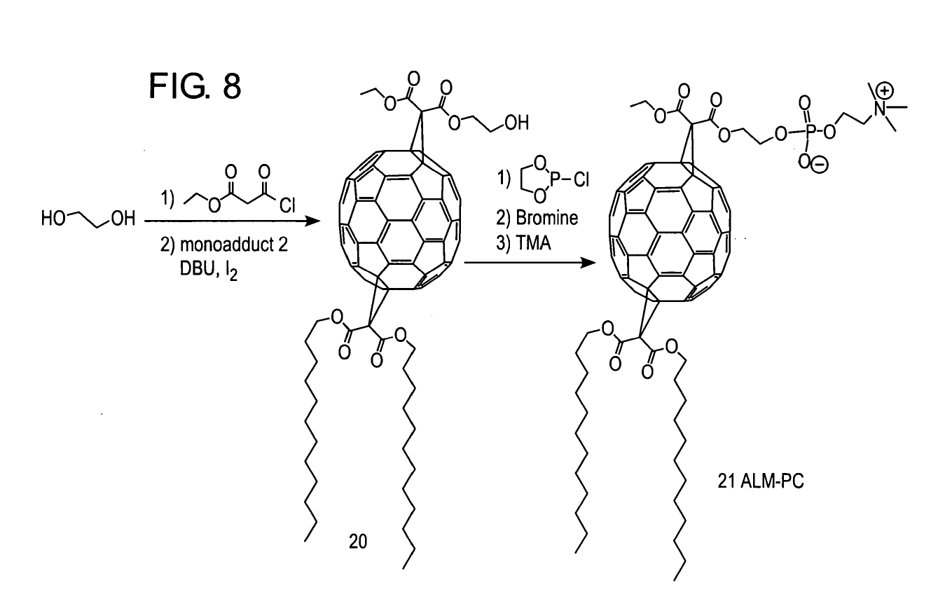

### Preparation of C₇₀-ALM-PC, Compounds 21 (ALM-PC in FIG. 8)

Step 1. Synthesis of ALM-Alcohol (20 in FIG. 8)

40 mmole ethylene glycol was dissolved in 50 mL dry THF, to which 12 mmole TEA was added and stirred under nitrogen. Then, 10 mmole ethyl malonyl chloride in 1 ml dry DCM, was slowly added in 10 minutes. The reaction mixture was stirred for additional 4 hours, then was washed with brine twice and the combined organic phase was dried over MgSO₄, filtered, and concentrated to 1-2 mL. Flash column with silica gel was used to purify the products with EA as the solvents. Yield: 50%. Proton and carbon NMR confirmed the structure. The obtained hydroxylethyl ethyl malonate was subjected to standard Bingel conditions described in above examples, and the product 8 was purified using flash column and characterized with NMR and AMLDI-MS.

Step 2. Synthesis of Compound 21

To a solution of ALM-alcohol 20 (0.7 mmol) in 20 mL THF cooled with salt-ice bath was added DIPEA (0.5 mL, 2.8 mmol) and ethylene chlorophosphite (0.2 mL, 2.1 mmol). The reaction mixture was stirred for 90 minutes and then neat bromine (0.1 mL, 2.1 mmol) was added. After 30 min, 5 mL water was added and stirred with the temperature slowly rising to RT after salt-ice bath was removed. Then 20 mL DCM was added and phase separated. The organic phase was rotavapored to dryness. The residue was then reconstituted in 1:1 DCM and isopropanol. 1 mL 40% trimethylamine (17 mmol) solution was added and stirred for 2 hours at 0° C. The mixture was stirred at RT for another 12 hours and concentrated. The residue was chromatographed on silica gel with DCM/EA/CH₃OH as the solvent system. The product was eluted as the third band after unreacted and intermediate fullerene compounds were removed. NMR shows singlet peak at 3.1-3.2 ppm corresponding to the three methyl groups on the quaternary amine.

### Example 2

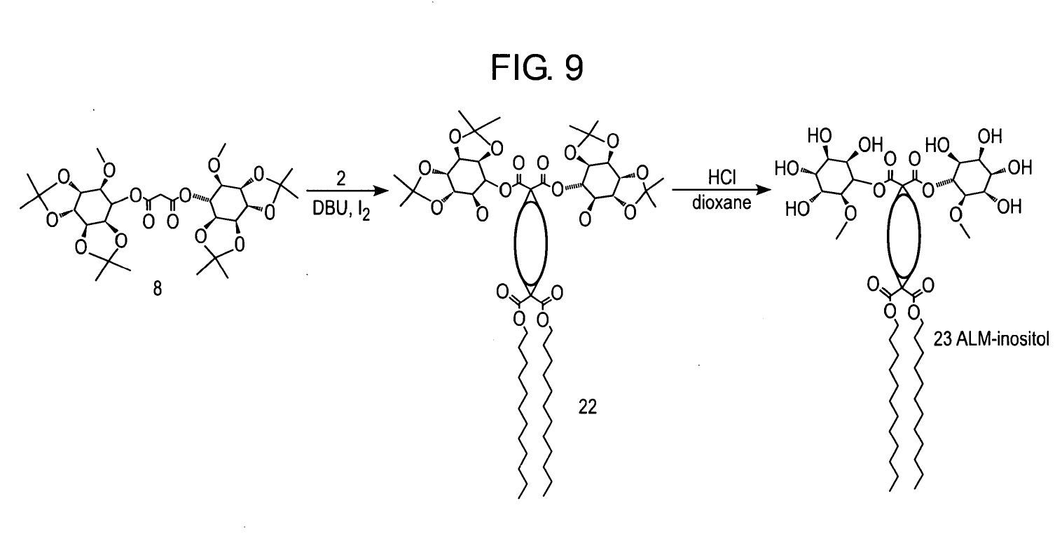

### Preparation of C₇₀-ALM-Inositol, Compound 23 (ALM Inositol in FIG. 9)

Step 1. Synthesis of ALM-Bis(Protected Inositol) (22 in FIG. 9)

127.8 mg (0.1 mmole) of C₇₀ monoadduct 2 (MW=1278) was dissolved in 60 mL toluene. Next, 0.1 mmole of the malonate 8 was added and the whole mixture was stirred, to which 0.1 mmole of iodine (MW=254 Da) was added. After stirred for 15 minutes, a 10 mL toluene solution of DBU (0.25 mmol, MW=151 Da) was added to the mixture over a period of 10 minutes, and stirred for 6 hours. When it is complete, the reaction mixture was concentrated to 10 mL and loaded to the top of a silica gel column for purification. Toluene was used first to elute unreacted C₇₀ monoadduct and then solvent was changed to DCM/EA to elute the product, which was then rotavaped and pumped under vacuum for overnight before NMR and MALDI-MS.

Step 2. Synthesis of Compound 23

50 mg of the obtained ALM-bis(protected inositol) 22 was dissolved in 2 mL chloroform and then 20 mL 4.0M hydrochloride dioxane solution was added. A few drops of water was added to keep starting materials un-precipitated in a homogenous solution, and stirred for 20 hours. Complete deprotection was achieved. Solvent were removed completely and dried under vacuum overnight to yield pure final product, with NMR and MALDI-MS data confirmed.

### Example 3

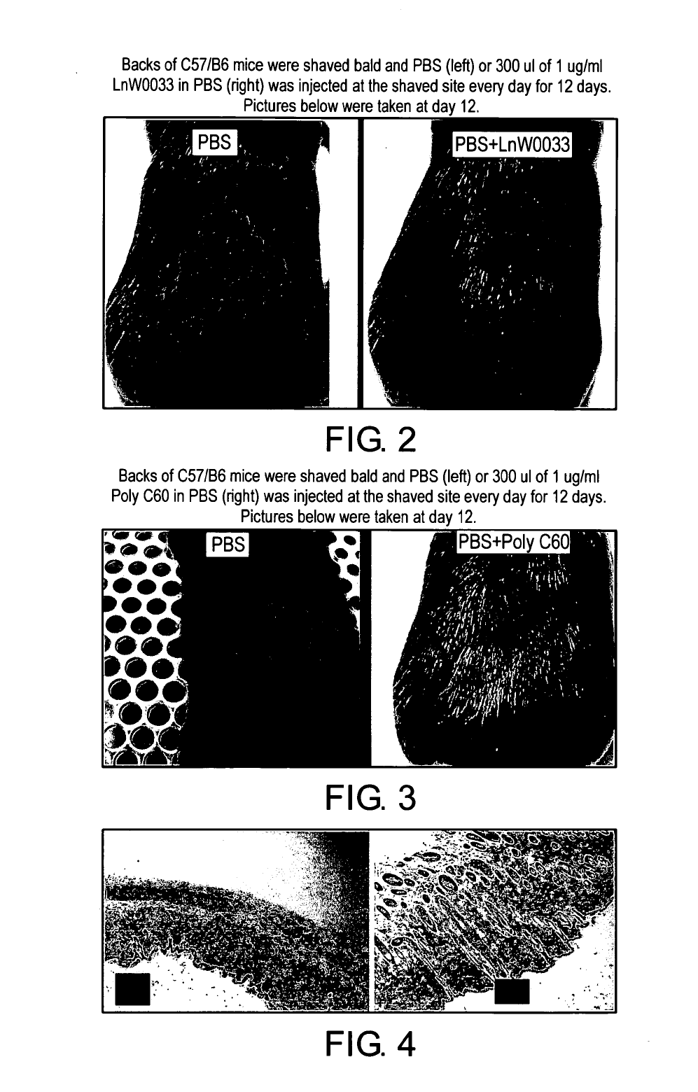

The backs of C57/B6 mice were shaved bald on the intracapsular part of the back. 300 μl of phosphate buffered saline (PBS) or 300 μl of 1 μg/ml of compound 5 (labelled LnW0033 in FIG. 2) in PBS was injected at the shaved site every day for 12 days. The photograph shown in FIG. 2 was taken at day 12 and shows the results of control (left) and fullerene-treated (right) mice.

Example 4

The backs of C57/B6 mice were shaved bald on the intracapsular part of the back. 300 μl PBS or 300 μl of 1 μg/ml Polyhydroxy-C₆₀ (C₆₀(OH)_{˜18-24;} BuckyUSA, Houston, Tex.; called “Poly C₆₀” in FIG. 3) in PBS was injected at the shaved site every day for 12 days. The photograph shown in FIG. 3 was taken at day 12 and shows the results of control (left) and fullerene-treated (right) mice.

The results shown in FIGS. 2 and 3 demonstrate that fullerenes administered at sites where hair loss has occurred promote significantly more hair growth as compared to controls.

### Example 5

300 μl of PBS or 3 μg of compound 5 in 300 μg of PBS was injected intradermally into different sections of the backs of SKH-1 bald mice every other day for 14 days. The mice were sacrificed and the affected area photodocumented before removing the skin and hair for histological analysis (FIG. 4). After fixing skin sections in 4% paraformaldahyde, sections were embedded in parafilm, sectioned, and stained with hemotoxylin/eosin. The average number of new hair follicles per mm² in fullerene treated was 86 and 24 in non-treated animals. The p value in this experiment was 0.003, which indicates a statistically significant increase in the number of new hair follicles in the treated vs. non-treated animals.

These results demonstrate that fullerenes induce hair follicle generation as compared to controls. On average the fullerene treated animals had approximately 50%-90% more new hair follicles than non-treated animals.

### Example 6

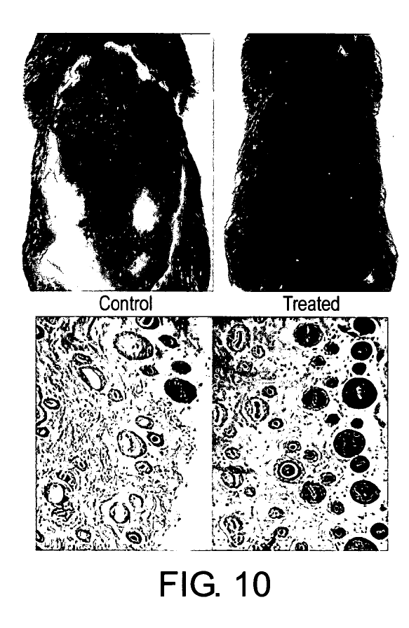

The backs of 7 week old C57/B1 mice were shaved after sedation and then treated with a commercial depilitant (thioglycolate). Two days later they were treated with 0.3 ml of a solution of DGME (control) or DGME containing 9 ug of compound 7 (C₇₀-TGA) applied topically. The mice were further treated every other day for a total of 14 days (7 treatments). At the conclusion the control and treated mice were photographed ((FIG. 10, top left and right panels, respectively) and processed to obtain histological slides (FIG. 10, bottom left and right panels, respectively). The number of hair follicles in a 1 cm reticle were counted by a technician who was blinded to the identity of the samples. There were 71±7 follicles in the slides prepared from the control animals and 93±11 follicles in those prepared from the treated animals (P value=0.0009).

While not wishing to be bound by theory, it is thought that excessive free radical generation (oxidative stress) is one mechanism leading ultimately to the loss of hair. Fullerene nanomaterials are very powerful antioxidants. We have shown that fullerene-based therapeutic formulations can enhance and stimulate hair growth.

1. A method for preventing hair loss and/or promoting hair growth, comprising administering to a subject in need thereof a therapeutically effective amount of a synthetically modified fullerene of the formula
Zₘ—F—Yₙ
wherein F is a fullerene of formula Cp or X@Cp, the fullerene having two opposing poles and an equatorial region;
Cp represents a fullerene cage having p carbon atoms, and X@Cp represents such a fullerene cage having a chemical group X within the cage.
Z and Y are positioned near respective opposite poles of Cp;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gi=1-3Hk=3-iN in which G and H are metal atoms.

2. The method of claim 1, wherein p is an even number between 60 and 120.

3. The method of claim 2, wherein p is an even number between 60 and 96.

4. The method of claim 3, wherein p is 60 or 70.

5. The method of claim 4, wherein p is 70.

6. The method of claim 1, wherein said synthetically modified fullerene is a prolate ellipsoid shaped fullerene having a major axis such that said poles are located at opposing ends of the major axis of the prolate ellipsoid fullerene.

7. The method of claim 1, wherein said synthetically modified fullerene is spheroid with opposing poles defined by an axis through opposing carbon rings.

8. A method for preventing hair loss and/or promoting hair growth, comprising administering to a subject in need thereof a therapeutically effective amount of a synthetically modified fullerene of the formula
Z(Cₚ)Y
wherein p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y.

9. The method of claim 8, wherein Cₚ is C₇₀.

10. The method of claim 8, wherein Z is a hydrophilic moiety.

11. The method of claim 10, wherein the synthetically modified fullerene is compound 5.

12. A method for preventing hair loss and/or promoting hair growth, comprising administering to a subject in need thereof a therapeutically effective amount of a synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃H₃₋ᵢN in which G and H are metal atoms.

13. The method of claim 12, wherein p is an even number between 60 and 120.

14. The method of claim 13, wherein p is an even number between 60 and 96.

15. The method of claim 14, wherein p is 60 or 70.

16. The method of claim 15, wherein p is 70.

17. The method of claim 12, wherein said synthetically modified fullerene is a prolate ellipsoid shaped fullerene having a major axis such that said poles are located at opposing ends of the major axis of the prolate ellipsoid fullerene.

18. The method of claim 12, wherein said synthetically modified fullerene is spheroid with opposing poles defined by an axis through opposing carbon rings.

19. The method of claim 12, wherein
Z′ comprises the formula A′ᵣB wherein A′ is a hydrophilic, lipophilic or amphiphilic chemical moiety, r=1-4, and B is a chemical linker connecting said A′ to the fullerene;
Y′ comprises the formula DE′ᵥ wherein E′ is a hydrophilic or amphiphilic chemical moiety and, v=1-4, and D is a chemical linker connecting the chemical moiety Y′ to the fullerene.

20. A method for preventing hair loss and/or promoting hair growth, comprising administering to a subject in need thereof a therapeutically effective amount of a synthetically modified fullerene of the formula
Z′(Cₚ)Y′
wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

21. The method of claim 20, wherein
(a) Z′ and Y′ are both amphiphilic;
(b) Z′ and Y′ are both hydrophilic;
(c) one of Z′ and Y′ is amphiphilic while the other is hydrophilic;
(d) Z′ is lipophilic and Y′ is hydrophilic; or
(e) Z′ is lipophilic and Y′ is amphiphilic.

22. The method of claim 21, wherein Z′ and Y′ are both hydrophilic.

23. The method of claim 22, wherein Cₚ=C₇₀.

24. A synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for use in preventing hair loss and/or promoting hair growth.

25. A synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₋₁₋₃Hₖ₌₃₋ᵢN in which G and H are metal atoms,
for preparation of a medicament for preventing hair loss and/or promoting hair growth.

26. A synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms, for use in preventing hair loss and/or promoting hair growth.

27. A synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for preparation of a medicament for preventing hair loss and/or promoting hair growth.

28. A method of preventing hair loss and/or promoting hair growth, comprising administering a therapeutically effective amount of a fullerene to a subject in need thereof.

29. The method of claim 28 wherein said fullerene is derivatized.

30. The method of claim 29, wherein said fullerene is water soluble.

31. The method of claim 30, wherein said fullerene is Polyhydroxy-C60.

32. A method for preventing hair loss and/or promoting hair growth, comprising administering to a subject in need thereof a therapeutically effective amount of a synthetically modified fullerene, wherein the synthetically modified fullerene is any one or more of the compounds shown in the present figures.

33. The method of claim 32, wherein the synthetically modified fullerene is selected from the group consisting of compound 5, compound 7, compound 10, compound 12, compound 13, compound 21, and compound 23.

34. The method of claim 1, wherein said fullerene is administered to an area of skin exhibiting or suspected of hair growth reduction or hair loss.

35. The method of claim 34, wherein said fullerene is injected at said area.

36. The method of claim 34, wherein said fullerene is administered topically.

37. The method of claim 1, wherein said subject is a human.

38. The method of claim 1, wherein said hair loss is related to androgenetic alopecia (AGA), alopecia areata, and/or chemotherapy or radiation therapy.

39. A compound selected from the group consisting of C₇₀-ALM-PC (compound 21) and compound C₇₀-ALM-inositol (compound 23).
